(19) United States
(12) Patent Application Publication
(10) Pub. No.: US 2014/0364438 A1
(43) Pub. Date: Dec. 11, 2014

(54) TRIAZOLOPYRIDINE DERIVATIVES AS A TYROSINE KINASE INHIBITOR

(71) Applicant: HANMI PHARM. CO., LTD., Hwaseong-si, Gyeonggi-do (KR)

(72) Inventors: Jae Yi Sim, Seoul (KR); Kyung Ik Lee, Anyang-si (KR); Ho Seok Kim, Hwaseong-si (KR); Tae Hee Ha, Hwaseong-si (KR); Kwee Hyun Suh, Suwon-si (KR)

(73) Assignee: HANMI PHARM. CO., LTD., Hwaseong-si, Gyeonggi-do (KR)

(21) Appl. No.: 14/376,562

(22) PCT Filed: Jan. 23, 2013
(86) PCT No.: PCT/KR2013/000539

§ 371 Date: Aug. 4, 2014

(30) Foreign Application Priority Data
Feb. 8, 2012 (KR) 10-2012-0012758

(51) Int. Cl. C07D 471/04; A61K 31/437; A61K 45/06; A61K 31/496

(52) U.S. Cl. CPC C07D 471/04; A61K 31/496; A61K 31/437; A61K 45/06
USPC 514/253.04; 544/362; 546/119; 514/303

(57) ABSTRACT

Provided is a novel triazolopyridine derivative having irreversible tyrosine kinase inhibiting activities, and a pharmaceutical composition comprising the same which can be useful for prevention or treatment of inflammatory diseases, autoimmune diseases, proliferative diseases or hyperproliferative diseases, immunologically mediated diseases, cancers or tumors.

### FIELD OF THE INVENTION

The present invention relates to novel triazolopyridine derivatives having irreversible tyrosine kinase inhibiting activities, and a pharmaceutical composition comprising the same as an active ingredient.

### BACKGROUND OF THE INVENTION

A protein kinase is an enzyme that modifies other proteins by chemically adding phosphate groups to a specific residue thereof via phosphorylation. The human genome contains about 500 protein kinase genes and they constitute about 2% of all human genes. In general, protein kinases can be classified into three types depending on their substrates: serine/threonine-specific protein kinases which phosphorylate serine and/or threonine residues, tyrosine-specific protein kinases which phosphorylate tyrosine residues, and protein kinases which phosphorylate tyrosine and serine/threonine residues. Protein kinases play a key role in mediation of signal transduction from a cell surface to a nucleus in response to a variety of extracellular stimuli. They regulate several physiological and pathological cellular phenomena, including cell division, proliferation, differentiation, apoptosis, cell mobility, mitogenesis, etc., and hence they are closely related with various diseases. Examples of such kinase-related diseases are: autoimmune disorders such as atopic dermatitis, asthma, rheumatoid arthritis, Crohn's disease, psoriasis, Crouzon syndrome, achondroplasia, and thanatophoric dysplasia; cancer such as prostate cancer, colorectal cancer, breast cancer, brain and throat cancer, leukemia and lymphoma; diabetes; restenosis; atherosclerosis; renal and hepatic fibrosis; myeloproliferative disorder and lymphoproliferative disorder; and eye disease. It is known that such diseases are caused directly or indirectly by interruption in kinase regulating mechanism such as mutation, overexpression or abnormal activation of kinase enzyme, and overproduction or underproduction of growth factors or cytokines which affect up-stream or down-stream signaling. Therefore, it is expected that such diseases may be prevented or treated by selectively inhibiting the mechanism of kinase, and thus various attempts have been made to discover an effective protein kinase inhibitor in the fields of medicine and chemistry.

Meanwhile, inflammation is a cause of disease such as rheumatoid arthritis, etc. Continuous attempts have been made to develop an effective medicine to treat inflammation despite the recent discovery of biological treatments. Various evidence has been found which support T-cells (or T-lymphocytes) and B-cells (or B-lymphocytes) play an important role in connection with the outbreak of inflammatory diseases, autoimmune diseases, proliferative or hyper-proliferative diseases and/or immunologically mediated diseases.

Such T-cells mediate signal transduction by receiving signals from antigen-presenting cells through T-cell receptor (TCR) located on the surface of the cell which activates various kinases such as Janus kinase (JAK) so as to forward the signal to effectors. In this regard, JAK proteins, as tyrosine kinases, may be activated by hematopoietic cytokine as well as interferon, and this process can regulate the activation of transcriptional regulators, STAT proteins. Therapeutic possibilities based on the inhibition (or promotion) of JAK/STAT pathway may provide a potent medication in the field of immunomodulation.

Among 4 types of JAK proteins, JAK3 is believed to be implicated in inflammation as it is expressed only in T-cells and activated by IL-2. Unlike JAK2 which participates in hemopoietic activity and red blood cell homeostasis or JAK1 which can be expressed in different types of tissues, JAK3 is mostly expressed in lymphocytes and plays a very important role in signaling by using various cytokines including IL-2, IL-4, IL-7, IL-9, IL-15 and the like, and therefore JAK3 is getting more attention in respect of side effects (Flanagan et al., Journal of Medicinal Chemistry, 53, 8468, 2010). According to animal studies, JAK3 plays an important role not only in maturation of B-cells and T-cells, but also in maintenance of functions of T-cells. Therefore, a JAK inhibitor, especially JAK3 inhibitor, may be useful for treatment of autoimmune disorders such as rheumatoid arthritis, psoriasis, atopic dermatitis, lupus, multiple sclerosis, Type I diabetes and diabetic complications, cancer, asthma, thyroid autoimmune disease, ulcerative colitis, Crohn's disease, Alzheimer's disease, leukemia, etc. as well as various conditions where immunosuppression is required, such as allograft rejection and xenotransplantation (Pesu M, Laurence A, Kishore N, et al., Immunol. Rev, 223, 132, 2008; Kawahara A, Minami Y, Miyazaki T, et al., Proc. Natl. Acad. Sci. USA, 92, 8724, 1995; Nosaka T, van Deursen J M A, Tripp R A, et al., Science, 270, 800, 1995; and Papageorgiou A C, Wikman L E K., et al., Trends Pharm. Sci., 25, 558, 2004)

Meanwhile, Bruton's tyrosine kinase (BTK) is a type of TEC-kinase family which plays an important role in activation of B-cells as well as signal transduction. In 1993, it was discovered that mutations in BTK are related with the major B-cell immune deficiency, i.e., X-linked Agammaglobulinemia (XLA) and mouse X-linked immunodeficiency (XID). It was also discovered that BTK is a nonreceptor protein tyrosine kinase (NRPTK) which participates in controlling signal transduction pathway, growth and differentiation of B-lymphocytes.

BTK is a key regulator of B-cell development, activation, signaling, and survival (Kurosaki, Curr. Op. Imm., 276-281, 2000; and Schaeffer and Schwartzberg, Curr. Op. Imm., 282-288, 2000]. In addition, BTK plays a role in a number of other hematopoietic cell signaling pathways, e.g., toll-like receptor (TLR)- or cytokine receptor-mediated TNF-α production in macrophages, IgE receptor (FcepsilonRi) signaling in mast cells, inhibition of Fas/APO-1 apoptotic signaling in B-lymphocytes, and collagen-stimulated platelet aggregation.

BTK participates in signal transduction pathways initiated by the binding of a variety of extracellular ligands to their cell surface receptors. After B-cell antigen receptor (BCR) ligation by antigen, BTK activation by the concerted actions of protein tyrosine kinases Lyn and Syk is required for induction of phospholipase C-γ2-mediated calcium mobilization (Kurosaki, T., Curr. Opin. Immunol., 9, 309-318, 1997). Therefore, inhibition of BTK can become a useful therapeutic option since it prevents the development of B-cell mediated diseases.

For instance, BTK deficient mice have been shown to be resistant to disease manifestations in collagen-induced arthritis, and BTK inhibitor is known to be effective against collagen-induced arthritis in mice dose-dependently (Jansson and Holmdahl, Clin. Exp. Immunol., 94, 459, 1993; and Pan et al., Chem. Med. Chem., 2, 58, 2007). Therefore, an effective BTK inhibitor may be useful for treatment of rheumatoid arthritis.

Further, inhibition of BTK activation can be useful for treatment of autoimmune disease and/or inflammatory disease and/or allergic disease, e.g., systemic lupus erythematosus (SLE), rheumatoid arthritis, psoriatic arthritis, osteoarthritis, juvenile arthritis, diabetes, myasthenia gravis, Hashimoto's thyroiditis, multiple sclerosis, ankylosing spondylitis, angiitis, inflammatory bowel disease, psoriasis, alopecia universalis, idiopathic thrombocytopenic purpura (ITP), myasthenia gravis, allergy, allergic conjunctivitis, allergic rhinitis, atopic dermatitis, and asthma, but not limited thereto. Also, it is known that BTK regulates apoptosis in cells, and hence inhibition of BTK activation can be used to treat B-cell lymphoma and leukemia as well.

As explained above, Janus kinases such as JAK3 and TEC kinases such as BTK play important roles in activation of T-cell and/or B-cell that are closely related with development of inflammatory diseases, autoimmune diseases, proliferative diseases or hyperproferative diseases and immunologically mediated diseases. Hence, development of an effective inhibitor of such kinases may lead to discovery of potent drug for treatment of various inflammatory diseases, autoimmune diseases, proliferative diseases or hyperproliferative diseases, and immunologically mediated diseases.

Currently, Tofacitinib (CP-690550), as an inhibitor of JAK3, is in development as an oral drug by Pfizer and a phase III trial is under way. PCI-32765 (Pharmacyclics), as an inhibitor of BTK, is in phase I clinical trial stage; however, it has been reported that the drug could activate a different target, accompanied by adverse side effects including skin rash and diarrhea. Therefore, there is a strong need for a novel drug which can inhibit Janus kinases and TEC kinases in a safe and effective manner.

### SUMMARY OF THE INVENTION

Therefore, it is an object of the present invention to provide a novel compound which inhibits kinases that are mostly expressed in abnormally activated lymphocytes (T-lymphocytes and/or B-lymphocytes) including Janus kinases such as JAK3 as well as TEC kinases such as BTK (Burton's tyrosine kinase), ITK (IL2-inducible T-cell kinase), BMX (bone marrow tyrosine kinase), RLK (resting lymphocyte kinase) and the like.

It is another object of the present invention to provide a pharmaceutical composition comprising the compound for prevention or treatment of inflammatory diseases, autoimmune diseases, proliferative diseases or hyperproliferative diseases, immunologically mediated diseases, cancers or tumors.

It is a further object of the present invention to provide a method for prevention and treatment of inflammatory diseases, autoimmune diseases, proliferative diseases or hyperproliferative diseases, immunologically mediated diseases, cancers, or tumors by using the compound.

It is a still further object of the present invention to provide a use of the compound for the manufacture of a medicament for preventing or treating inflammatory diseases, autoimmune diseases, proliferative diseases or hyperproliferative diseases, immunologically mediated diseases, cancers, or tumors.

In accordance with one aspect of the present invention, there is provided a compound of formula (I) or a pharmaceutically acceptable salt thereof:

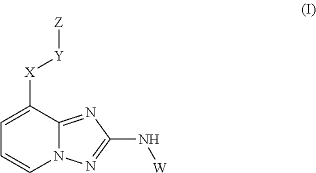

wherein,

X is O, NH, CH₂, S, SO or SO₂;

Y is phenyl or pyridyl;

Z is

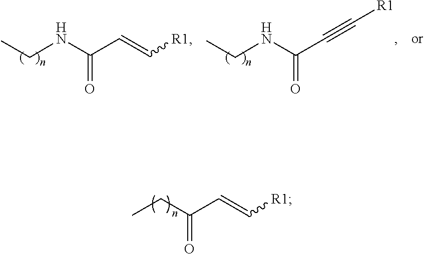

n is an integer ranging from 0 to 4;

R1 is each independently hydrogen, C₁₋₆alkoxy or di(C₁₋₆alkyl)aminomethyl; and

W is phenyl, pyridyl, or phenyl substituted with one or more substituents selected from the group consisting of hydrogen, halogen, hydroxy, amino, C₁₋₆alkylamino, C₁₋₆alkylheterocyclylamino, di(C₁₋₆alkyl)aminoC₁₋₆alkyl, heterocycle, hydroxy heterocycle, C₁₋₆alkylheterocycle, hydroxyC₁₋₆alkylheterocycle, C₁₋₆alkoxyC₁₋₆alkylheterocycle, heterocyclylcarbonyl, and heterocyclylC₁₋₆alkylcarbonyl, wherein the heterocycle is a saturated 3- to 8-membered monocyclic hetero ring independently containing one or more of heteroatoms selected from N, O and S.

In accordance with another aspect of the present invention, there is provided a pharmaceutical composition for preventing or treating inflammatory diseases, autoimmune diseases, proliferative diseases or hyperproliferative diseases, immunologically mediated diseases, cancers, or tumors, which comprises the compound of formula (I) or a pharmaceutically acceptable salt thereof.

In accordance with a further aspect of the present invention, there is provided a method for preventing or treating inflammatory diseases, autoimmune diseases, proliferative diseases or hyperproliferative diseases, immunologically mediated diseases, cancers, or tumors in an animal, comprising the step of administering to the animal an effective amount of the compound of formula (I) or a pharmaceutically acceptable salt thereof.

In accordance with a still further aspect of the present invention, there is provided a use of the compound of formula (I) or a pharmaceutically acceptable salt thereof for the manufacture of a medicament for preventing or treating inflammatory diseases, autoimmune diseases, proliferative diseases or hyperproliferative diseases, immunologically mediated diseases, cancers, or tumors.

The novel triazolopyridine derivatives in accordance with the present invention can selectively and effectively inhibit kinases that are mostly expressed in abnormally activated lymphocytes (T-lymphocytes and/or B-lymphocytes) including Janus kinases such as JAK3 as well as TEC kinases such as BTK, ITK, BMX and RLK and the like. Therefore, the novel triazolopyridine derivatives as a tyrosine kinase inhibitor in accordance with the present invention may be useful for prevention or treatment of diseases that are mediated by abnormally activated T-lymphocytes, B-lymphocytes or both, such as inflammatory diseases, autoimmune diseases, proliferative diseases or hyperproliferative diseases, immunologically mediated diseases, cancers, or tumors.

### DETAILED DESCRIPTION OF THE INVENTION

In formula (I), specific examples of substituent W may be selected from the group consisting of W1 to W18, preferably W2, W4, W9, W12 or W17, but not limited thereto.

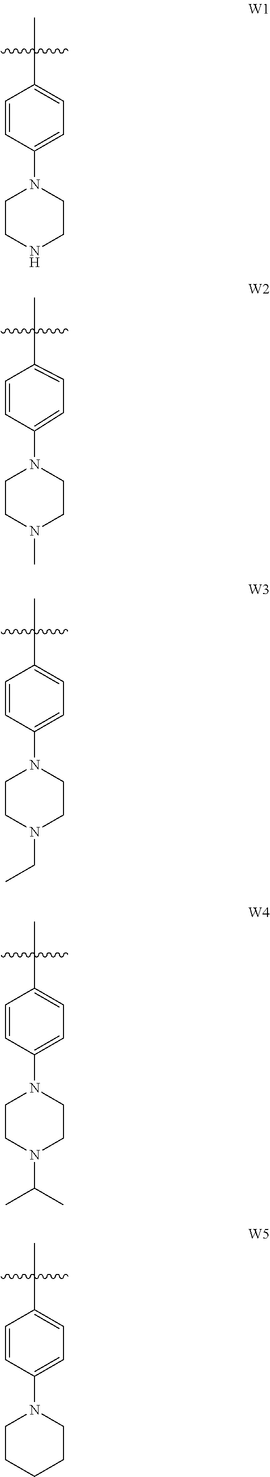 [SMILES: *c1ccc(N2CCNCC2)cc1 ; *c1ccc(N2CCN(C)CC2)cc1 ; *c1ccc(N2CCN(CC)CC2)cc1 ; *c1ccc(N2CCN(C(C)C)CC2)cc1 ; *c1ccc(N2CCCCC2)cc1]

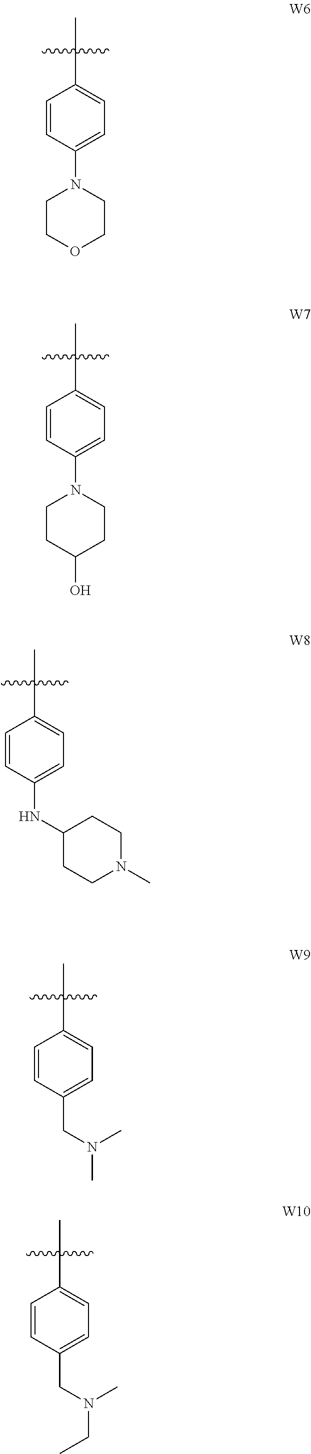 [SMILES: *c1ccc(N2CCOCC2)cc1 ; *c1ccc(N2CCC(O)CC2)cc1 ; *c1ccc(NC2CCN(C)CC2)cc1 ; *c1ccc(CN(C)C)cc1 ; *c1ccc(CN(C)CC)cc1]

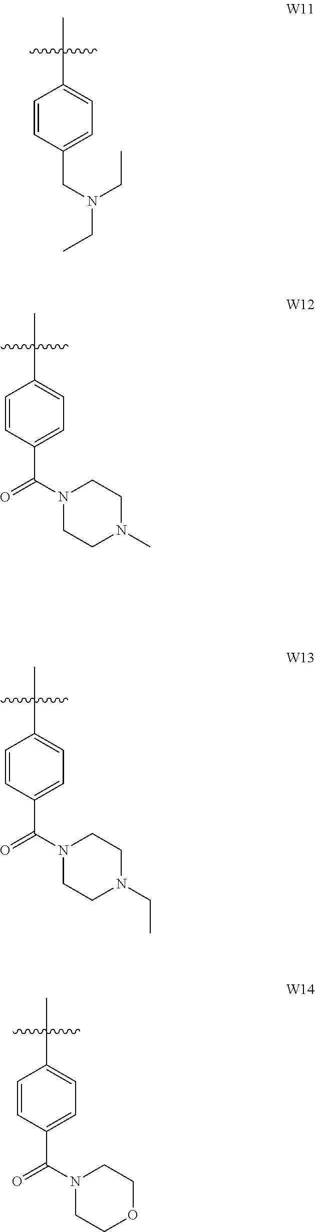 [SMILES: *c1ccc(CN(CC)CC)cc1 ; *c1ccc(C(=O)N2CCN(C)CC2)cc1 ; *c1ccc(C(=O)N2CCN(CC)CC2)cc1 ; *c1ccc(C(=O)N2CCOCC2)cc1]

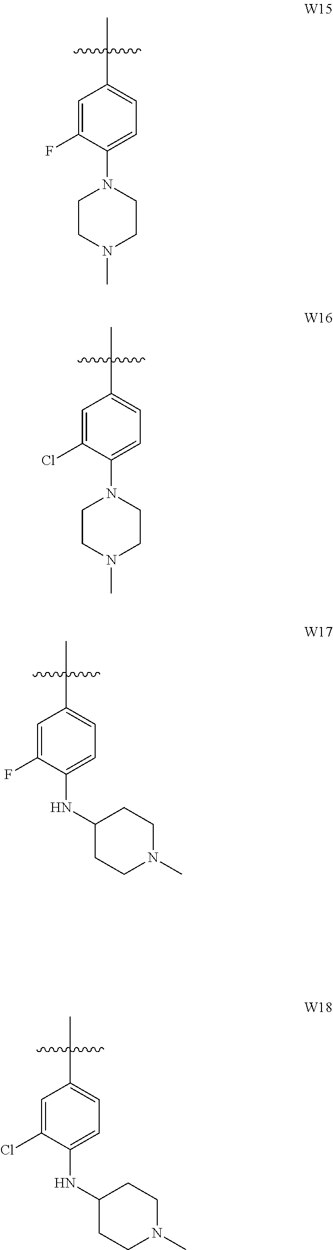 [SMILES: *c1ccc(N2CCN(C)CC2)c(F)c1 ; *c1ccc(N2CCN(C)CC2)c(Cl)c1 ; *c1ccc(NC2CCN(C)CC2)c(F)c1 ; *c1ccc(NC2CCN(C)CC2)c(Cl)c1]

The examples of the compounds in accordance with the present invention are as follows:
N-(3-(2-(4-(4-methylpiperazin-1-yl)phenylamino)[1,2,4]triazolo[1,5-a]pyridin-8-yloxy)phenyl)acrylamide;
N-(3-(2-(3-fluoro-4-(1-methylpiperidin-4-ylamino)phenylamino)-[1,2,4]triazolo[1,5-a]pyridin-8-yloxy)phenyl)acrylamide;
N-(3-(2-(4-((dimethylamino)methyl)phenylamino)-[1,2,4]triazolo[1,5-a]pyridin-8-yloxy)phenyl)acrylamide;
N-(3-(2-(4-(4-methylpiperazin-1-carbonyl)phenylamino)-[1,2,4]triazolo[1,5-a]pyridin-8-yloxy)phenyl)acrylamide; and
N-(3-(2-(4-(4-isopropylpiperazin-1-yl)phenylamino)-[1,2,4]triazolo[1,5-a]pyridin-8-yloxy)phenyl)acrylamide.

The compound of formula (I) of the present invention may be prepared by the method shown in Reaction Scheme I as shown below:

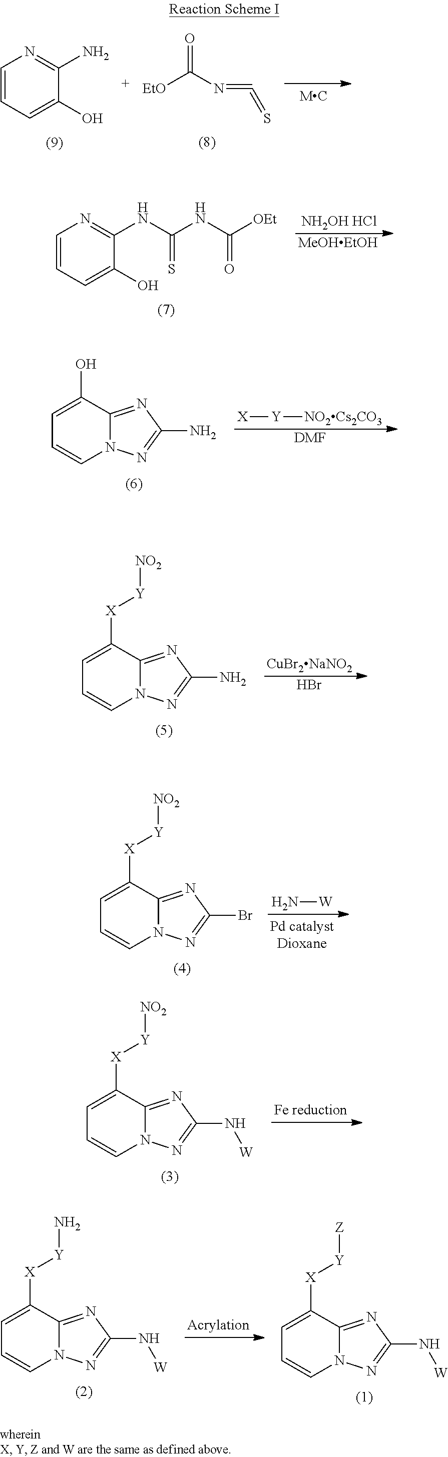

The reaction processes are exemplified in the following stepwise reaction.

The compound of formula (9) is, for example, subjected to a condensation reaction with the compound of formula (8) under dichloromethane condition to yield a condensed compound of formula (7). Then, hydroxylamine hydrochloride and diisopropylethylamine may be added to a solvent such as a mixed solvent of methanol and ethanol, followed by addition of the compound of formula (7) prepared above to obtain a compound of formula (6).

Next, the compound of formula (6) is allowed to react with X—Y—NO₂ (e.g., 3-fluoronitrobenzene) in an organic solvent such as N,N-dimethylformamide, N,N-dimethylacetamide or N-methylpyrrolidine in the presence of an inorganic base such as cesium carbonate, sodium carbonate or potassium carbonate at 140 to 150° C. with stirring to obtain a compound of formula (5) containing a nitro group. Copper bromide and bromic acid are added to the compound of formula (5), followed by dropwise addition of an aqueous solution of sodium nitrite at −10 to 0° C. to obtain a compound of formula (4) containing a bromine group.

The compound of formula (4) prepared above may be reacted with W—NH₂ in an organic solvent such as 1,4-dioxane in the presence of a palladium catalyst or trifluoroacetic acid at 100 to 110° C. for 8 hours with stirring to obtain a compound of formula (3) containing a W—NH₂ group.

The nitro group of the compound of formula (3) may be converted to an amino group by subjecting the compound to an iron-mediated reduction reaction or a hydrogenation reaction using palladium/carbon as a catalyst to obtain an aniline compound of formula (2).

Subsequently, the compound of formula (2) may be allowed to react with an acryloyl chloride substituted with R1 in an organic solvent such as dichloromethane or tetrahydrofuran, or a mixed solvent such as an aqueous solution of 50% tetrahydrofuran in the presence of an inorganic base such as sodium bicarbonate or an organic base such as triethylamine or diisopropylethylamine at a low temperature ranging from −10° C. to 10° C.; or with an acrylic acid substituted with R1 by employing a binder such as 1-ethyl-3-(3-dimethylaminopropyl)carbodiimide (EDCI) or 2-(1H-7-azabenzotriazol-1-yl)-1,1,3,3-tetramethyl uronium hexafluorophosphate methanaminium (HATU) in pyridine, to obtain a desired compound of formula (1) of the present invention containing an acrylamide group.

The compound of formula (I) in the present invention may also form a pharmaceutically acceptable inorganic or organic acid addition salts. Examples of such salts are acid addition salts formed by acids such as hydrochloric acid, hydrobromic acid, sulfuric acid, phosphoric acid, nitric acid, acetic acid, glycolic acid, lactic acid, pyruvic acid, malonic acid, succinic acid, glutaric acid, fumaric acid, malic acid, mandelic acid, tartaric acid, citric acid, ascorbic acid, palmitic acid, maleic acid, hydroxymaleic acid, benzoic acid, hydroxybenzoic acid, phenylacetic acid, cinnamic acid, salicylic acid, methanesulfonic acid, benzenesulfonic acid, toluenesulfonic acid and the like.

Specifically, the pharmaceutically acceptable salt in the present invention can be prepared by dissolving the compound of formula (I) in a water-miscible organic solvent, e.g., acetone, methanol, ethanol or acetonitrile, followed by adding an organic or inorganic acid, and filtering the precipitated crystal. Also, it may be prepared by removing a solvent or an excessive amount of acid from the acid-added reaction mixture under reduced pressure, followed by drying the residue, or conducting reduction using a different organic solvent, and then filtering the precipitated salt.

The compound of formula (I) or a pharmaceutically acceptable salt thereof in the present invention may be in the form of solvates or hydrates, and such compounds are also included within the scope of the present invention.

The compound of formula (I) or a pharmaceutically acceptable salt thereof in the present invention can selectively and effectively inhibit a protein kinase. In one embodiment, such compound can selectively and effectively inhibit kinases that are mostly expressed in abnormally activated lymphocytes (T-lymphocytes and/or B-lymphocytes) including Janus kinase 3 (JAK3), Bruton's tyrosine kinase (BTK), IL-2 inducing T-cell kinase (ITK), resting lymphocyte kinase (RLK) and bone marrow tyrosine kinase (BMX), and thus, may be useful for prevention or treatment of diseases that are mediated by abnormally activated B-lymphocytes, T-lymphocytes or both, such as inflammatory diseases, autoimmune diseases, proliferative diseases or hyperproliferative diseases, immunologically mediated diseases, cancers, or tumors.

Therefore, the present invention provides a pharmaceutical composition for preventing or treating inflammatory diseases, autoimmune diseases, proliferative diseases or hyperproliferative diseases, immunologically mediated diseases, cancers, or tumors, which comprises the compound of formula (I) or its pharmaceutically acceptable salt as an active ingredient.

Examples of said inflammatory diseases, autoimmune diseases, proliferative diseases or hyperproliferative diseases, or immunologically mediated diseases may be selected from the group consisting of: arthritis, rheumatoid arthritis, spondyloarthropathy, gouty arthritis, osteoarthritis, juvenile arthritis, other arthritic conditions, lupus, systemic lupus erythematosus (SLE), skin-related diseases, psoriasis, eczema, dermatitis, atopic dermatitis, pain, pulmonary disorder, lung inflammation, adult respiratory distress syndrome (ARDS), pulmonary sarcoidosis, chronic pulmonary inflammatory disease, chronic obstructive pulmonary disease (COPD), cardiovascular disease, artherosclerosis, myocardial infarction, congestive heart failure, cardiac reperfusion injury, inflammatory bowel disease, Crohn's disease, ulcerative colitis, irritable bowel syndrome, asthma, Sjogren's syndrome, autoimmune thyroid disease, urticaria, multiple sclerosis, scleroderma, allograft rejection, xenotransplantation, idiopathic thrombocytopenic purpura (ITP), Parkinson's disease, Alzheimer's disease, diabetic associated disease, inflammation, pelvic inflammatory disease, allergic rhinitis, allergic bronchitis, allergic sinusitis, leukemia, lymphoma, B-cell lymphoma, T-cell lymphoma, myeloma, acute lymphoid leukemia (ALL), chronic lymphoid leukemia (CLL), acute myeloid leukemia (AML), chronic myeloid leukemia (CML), hairy cell leukemia, Hodgkin's disease, non-Hodgkin's lymphoma, multiple myeloma, myelodysplastic syndrome (MDS), myeloproliferative neoplasms (MPN), diffuse large B-cell lymphoma and follicular lymphoma, but not limited thereto.

Further, examples of said cancer and tumor may be selected from the group consisting of liver cancer, hepatocellular carcinoma, thyroid cancer, colorectal cancer, testicular cancer, bone cancer, oral cancer, basal cell carcinoma, ovarian cancer, brain tumor, gallbladder carcinoma, biliary tract cancer, head and neck cancer, vesical carcinoma, tongue cancer, esophageal cancer, glioma, glioblastoma, renal cancer, malignant melanoma, gastric cancer, breast cancer, sarcoma, pharynx carcinoma, uterine cancer, cervical cancer, prostate cancer, rectal cancer, pancreatic cancer, lung cancer, skin cancer and other solid tumor, but not limited thereto.

The compound of formula (I) or a pharmaceutically acceptable salt thereof in the present invention may be used in combination with other drugs to enhance efficacy in treatment of inflammatory diseases, autoimmune diseases, proliferative diseases or hyperproliferative diseases, or immunologically mediated diseases.

Examples of the drug which may be used in combination with the inventive compound or a pharmaceutically acceptable salt thereof for treatment of inflammatory diseases, autoimmune diseases, proliferative diseases or hyperproliferative diseases, or immunologically mediated diseases are one or more of drugs selected from the group consisting of steroids (prednisone, prednisolone, methylprednisolone, cortisone, hydroxycortisone, betamethasone, dexamethasone, etc.), methotrexate, lefluonomide, anti-TNFα agents (etanercept, infliximab, adalimumab, etc.), calcineurin inhibitors (tacrolimus, pimecrolimus, etc.) and anti-histamines (diphenhydramine, hydroxyzine, loratadine, ebastine, ketotifen, cetirizine, levocetirizine, fexofenadine, etc.), but not limited thereto.

Examples of the drug which may be used in combination with the inventive compound or its pharmaceutically acceptable salt for treatment of cancers or tumors include one or more selected from the group consisting of: cell signal transduction inhibitors (glivec, iressa, tarceva, etc.), mitosis inhibitors (vincristine, vinblastine, etc.), alkylating agents (cyclophosphamide, thiotepa, busulfan, etc.), anti-metabolites (tagafur, methotrexate, gemcitabine, etc.), topoisomerase inhibitors (irinotecan, topotecan, amsacrine, etoposide, teniposide, etc.), immunotherapeutic agents (interferon α, β, γ, interleukin, etc.) and anti-hormonal agents (tamoxifen, leuprorelin, anastrozole, etc.), but not limited thereto.

The inventive compound or a pharmaceutically acceptable salt thereof may be administered orally or parenterally as an active ingredient in an effective amount ranging from about 0.1 to 2,000 mg/day, preferably 1 to 1,000 mg/day, 1 to 4 times daily or on/off schedule in case of a human (of approximately 70 kg body weight) in a single dose or in divided doses. The dosage of the active ingredient may be adjusted in light of various relevant factors such as the condition of the subject to be treated, type and seriousness of illness, administration rate, and opinion of doctor. In certain cases, an amount less than the above dosage may be suitable. An amount greater than the above dosage may be used unless it causes deleterious side effects and such amount can be administered in divided doses per day.

The pharmaceutical composition of the present invention may typically comprise pharmaceutically acceptable additives, carriers or excipients. The pharmaceutical composition of the present invention may be formulated in accordance with conventional methods, and may be prepared in the form of oral formulations such as tablets, pills, powders, capsules, syrups, emulsions, microemulsions and others, or parenteral formulations such as intramuscular, intravenous or subcutaneous administrations.

For oral formulations, carriers or additives such as cellulose, calcium silicate, corn starch, lactose, sucrose, dextrose, calcium phosphate, stearic acid, magnesium stearate, calcium stearate, gelatin, talc, surfactants, suspending agents, emulsifiers, diluents, and others may be used. For injectable formulations, carriers or additives such as water, saline, glucose solution, glucose solution analogs, alcohols, glycols, ethers (e.g., polyethylene glycol 400), oils, fatty acids, fatty acid esters, glycerides, surfactants, suspending agents, emulsifiers, and others may be used.

Also, the present invention provides a method for preventing or treating inflammatory diseases, autoimmune diseases, proliferative diseases or hyperproliferative diseases, immunologically mediated diseases, cancers, or tumors in an animal, comprising the step of administering to the animal an effective amount of the compound of formula (I) or its pharmaceutically acceptable salt.

The present invention provides a use of the compound of formula (I) or a pharmaceutically acceptable salt thereof for the manufacture of a medicament for preventing or treating inflammatory diseases, autoimmune diseases, proliferative diseases or hyperproliferative diseases, immunologically mediated diseases, cancers, or tumors.

The compound of formula (I) of the present invention may be used for the study of biological and pathological phenomena of a kinase, the study of intracellular signaling pathway mediated by a kinase as well as comparative evaluation with new kinase inhibitors.

### EXAMPLES

The following Examples are provided to illustrate preferred embodiments of the present invention, and are not intended to limit the scope of the present invention.

### Example 1

### Preparation of N-(3-(2-(4-(4-methylpiperazin-1-yl)phenylamino)-[1,2,4]triazolo[1,5-a]pyridin-8-yloxy)phenyl)acrylamide

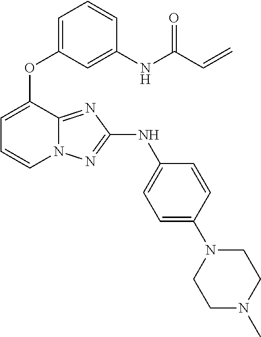 [SMILES: C=CC(=O)Nc1cccc(Oc2cccn3nc(Nc4ccc(N5CCN(C)CC5)cc4)nc23)c1]

### Step 1) Preparation of N-(3-hydroxy-2-pyridinyl)-N′-carboethoxy-thiourea

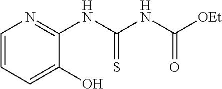 [SMILES: CCOC(=O)NC(=S)Nc1ncccc1O]

Dichloromethane (100 mL) was added to 2-amino-3-hydroxypyridine (10.0 g, 0.091 mol). The reaction solution was cooled to 0° C., and ethoxycarbonyl isothiocyanate (11.3 mL, 0.1 mol) was added dropwise thereto. The temperature of the mixture was raised to room temperature and the mixture was stirred for 12 hours. A solid formed was cooled to 0° C., washed with 20 mL of dichloromethane, and filtered under reduced pressure. The solid thus obtained was dried under reduced pressure to obtain the title compound (8.4 g, yield: 38%).

### Step 2) Preparation of 2-amino-[1,2,4]triazolo[1,5-a]pyridin-8-ol

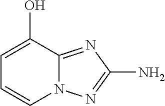

A mixed solvent (30 mL) of ethanol and methanol (1:1) was added to hydroxylamine hydrochloride (4.6 g, 0.066 mol) at room temperature. Diisopropylethylamine (11.6 mL, 0.066 mol) was added to the mixture, followed by stirring for 1 hour. The compound obtained in Step 1 (8.4 g, 0.035 mol) was added to the reaction solution, followed by refluxing for 2 hours at 80° C. or higher. The reaction solution was cooled to 0° C., stirred for 1 hour, and a solid formed was washed with 20 mL of distilled water and filtered under reduced pressure. The solid thus obtained was dried under reduced pressure to obtain the title compound (3.2 g, yield: 54%).

¹H-NMR (300 MHz, DMSO-d₆) δ 5.80 (s, 2H), 6.68 (m, 2H), 8.01 (d, 1H).

### Step 3) Preparation of 8-(3-nitrophenoxy)-[1,2,4]triazolo[1,5-a]pyridin-2-amine

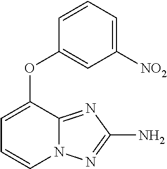 [SMILES: Nc1nc2c(Oc3cccc([N+](=O)[O-])c3)cccn2n1]

N,N-dimethylformamide (30 mL) was added to the compound obtained in Step 2 (3.2 g, 0.021 mol). 3-fluoronitrobenzene (2.7 mL, 0.026 mol) and cesium carbonate (13.9 g, 0.043 mol) was added to the reaction solution. The resulting mixture was stirred for 6 hours at 150° C., and then washed with dichloromethane, distilled water and an aqueous solution of ammonium chloride. The organic layer was separated, dried over anhydrous sodium sulfate, filtered and distilled under reduced pressure. The resulting residue was separated by column chromatography (dichloromethane:methanol=60:1 (v:v)) to obtain the title compound (1.7 g, yield: 30%).

¹H-NMR (300 MHz, DMSO-d₆) δ 6.14 (s, 2H), 6.94 (t, 1H), 7.35 (d, 2H), 7.47 (m, 1H), 7.64 (m 2H), 7.96 (d, 1H), 8.52 (d, 1H).

### Step 4) Preparation of 2-bromo-8-(3-nitrophenoxy)-[1,2,4]triazolo[1,5-a]pyridine

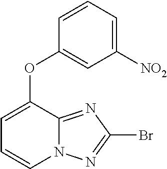 [SMILES: O=[N+]([O-])c1cccc(Oc2cccn3nc(Br)nc23)c1]

Bromic acid (17 mL, 47˜49%) was added to a mixture of the compound obtained in Step 3 (1.7 g, 0.006 mol) and copper bromide (0.42 g, 0.002 mol). The reaction solution was cooled to 0° C., and a solution prepared by dissolving sodium nitrite (0.52 g, 0.008 mol) in distilled water (3.5 mL), was added slowly dropwise thereto. The reaction solution was stirred for 15 hours at room temperature, and then washed with dichloromethane, distilled water and an aqueous solution of ammonium chloride. The organic layer was separated, dried over anhydrous sodium sulfate, filtered and distilled under reduced pressure. The resulting residue was separated by column chromatography (dichloromethane:methanol=40:1 (v:v)) to obtain the title compound (1.8 g, yield: 86%).

¹H-NMR (300 MHz, DMSO-d₆) δ 7.10 (m 2H), 7.48 (m, 1H), 7.60 (t, 1H), 7.90 (m, 1H), 8.06 (dd, 1H), 8.43 (dd, 1H).

### Step 5) Preparation of N-(4-(4-methylpiperazin-1-yl)phenyl-8-(3-nitrophenoxy)-[1,2,4]triazolo[1,5-a]pyridin-2-amine

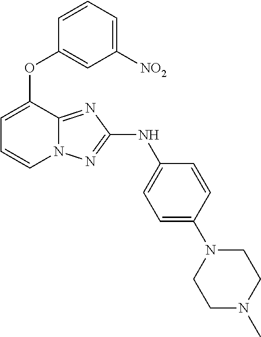 [SMILES: CN1CCN(c2ccc(Nc3nc4c(Oc5cccc([N+](=O)[O-])c5)cccn4n3)cc2)CC1]

1,4-dioxane (30 mL) was added to a mixture of the compound obtained in Step 4 (1.8 g, 0.005 mol) and 4-(4-methylpiperazin-1-yl)aniline (1.03 g, 0.005 mol). Tris(dibenzylideneacetone)dipalladium(0) (0.49 g, 0.001 mol) and 2,2′-bis(diphenylphosphino)-1,1′-binaphthyl (0.33 g, 0.001 mol) were added to the mixture, followed by stirring for 5 minutes at room temperature. Cesium carbonate (3.5 g, 0.011 mol) was added to the reaction mixture, followed by stirring for 8 hours at 100° C. The reaction mixture was cooled to room temperature, filtered through a Celite filter, and the filtrate was diluted with dichloromethane and washed with water. The organic layer was separated, dried over anhydrous sodium sulfate, filtered and distilled under reduced pressure. The resulting residue was separated by column chromatography (dichloromethane:methanol=20:1 (v:v)) to obtain the title compound (0.91 g, yield: 38%).

¹H-NMR (300 MHz, DMSO-d₆) δ 2.31 (s, 3H), 2.62 (m, 4H), 3.10 (m, 4H), 6.97 (m, 3H), 7.23 (d, 1H), 7.44 (m, 3H), 7.58 (t, 1H), 7.85 (m, 1H), 8.00 (m, 1H), 8.41 (d, 1H).

### Step 6) Preparation of 8-(3-aminophenoxy)-N-(4-(4-methylpiperazin-1-yl)phenyl)-[1,2,4]triazolo[1,5-a]pyridin-2-amine

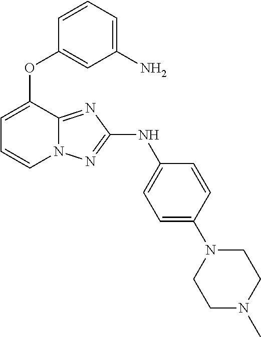 [SMILES: CN1CCN(c2ccc(Nc3nc4c(Oc5cccc(N)c5)cccn4n3)cc2)CC1]

Iron (0.57 g, 0.010 mol) and 12 N aqueous solution of hydrochloric acid (68 μL, 0.001 mol) were diluted in an aqueous solution of 50% ethanol, followed by stirring for 1 hour at 100° C. The compound obtained in Step 5 (0.91 g, 0.002 mol) was dissolved in an aqueous solution of 50% ethanol (10 mL), added to the reaction flask containing activated iron, followed by stirring for 1 hour at 100° C. The reaction mixture was filtered through a Celite filter so as to remove iron, and the filtrate was distilled under reduced pressure. The residue was diluted with dichloromethane, and washed with an aqueous solution of saturated sodium bicarbonate. The organic layer was separated, dried over anhydrous sodium sulfate, filtered and distilled under reduced pressure. The resulting residue was separated by column chromatography (dichloromethane:methanol=10:1 (v:v)) to obtain the title compound (0.76 g, yield: 90%).

¹H-NMR (300 MHz, DMSO-d₆) δ 2.21 (s, 3H), 2.44 (m, 4H), 3.02 (m, 4H), 5.22 (s, 2H), 6.18 (m, 2H), 6.32 (m, 1H), 6.99 (m, 4H), 7.12 (d, 1H), 7.52 (d, 2H), 8.57 (d, 1H), 9.38 (s, 1H).

### Step 7) Preparation of N-(3-((2-((4-(4-methylpiperazin-1-yl)phenyl)amino)-[1,2,4]triazolo[1,5-a]pyridin-8-yl)oxy)phenyl)acrylamide

Tetrahydrofuran (10 mL) and distilled water (2 mL) were added to the compound obtained in Step 6 (0.76 g, 0.002 mol) and sodium bicarbonate (0.46 g, 0.006 mol). Acryloyl chloride (0.18 mL, 0.002 mol) was added slowly dropwise to the reaction solution at 0° C., followed by stirring for 2 hours at room temperature. The reaction mixture was diluted with dichloromethane and then washed with an aqueous solution of saturated sodium bicarbonate. The organic layer was separated, dried over anhydrous sodium sulfate, filtered and distilled under reduced pressure. The resulting residue was separated by column chromatography (dichloromethane:methanol=10:1 (v:v)) to obtain the title compound (0.34 g, yield: 40%).

¹H-NMR (300 MHz, DMSO-d₆) δ 2.22 (s, 3H), 2.50 (m, 4H), 3.03 (m, 4H), 5.73 (dd, 1H), 6.23 (dd, 1h), 6.33 (m, 1H), 6.89 (m, 4H), 7.38 (m, 5H), 8.65 (d, 1H), 9.40 (s, 1H), 10.19 (s, 1H).

### Example 2

### Preparation of N-(3-(2-(3-fluoro-4-(1-methylpiperidin-4-ylamino)phenylamino)-[1,2,4]triazolo[1,5-a]pyridin-8-yloxy)phenyl)acrylamide

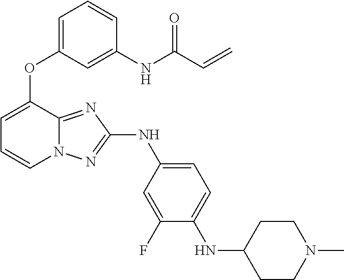 [SMILES: C=CC(=O)Nc1cccc(Oc2cccn3nc(Nc4ccc(NC5CCN(C)CC5)c(F)c4)nc23)c1]

The procedures of Steps 5, 6 and 7 of Example 1 were repeated in sequence, except for using the compound obtained in Step 4 (0.21 g, about 0.001 mol) and 2-fluoro-N¹-(1-methylpiperidin-4-yl)benzene-1,4-diamine (0.14 g, 0.001 mol) to obtain the title compound (0.1 g, yield: 32%).

¹H-NMR (300 MHz, DMSO-d₆) δ 1.43 (m, 1H), 1.82 (m, 2H), 1.95 (m, 2H), 2.15 (s, 3H), 2.72 (m, 2H), 3.13 (m, 1H), 4.53 (d, 1H), 5.73 (dd, 1H), 6.24 (dd, 1H), 6.35 (dd, 1H), 6.79 (m, 2H), 6.99 (t, 1H), 7.12 (dd, 1H), 7.40 (m, 5H), 8.66 (d, 1H), 9.45 (s, 1H), 10.22 (s, 1H).

### Example 3

### Preparation of N-(3-(2-(4-((dimethylamino)methyl)phenylamino)-[1,2,4]triazolo[1,5-a]pyridin-8-yloxy)phenyl)acrylamide

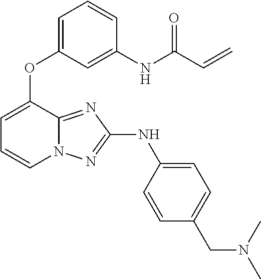 [SMILES: C=CC(=O)Nc1cccc(Oc2cccn3nc(Nc4ccc(CN(C)C)cc4)nc23)c1]

The procedures of Steps 5, 6 and 7 of Example 1 were repeated in sequence, except for using the compound obtained in Step 4 (0.45 g, 0.001 mol) and 4-((dimethylamino)methyl)aniline (0.2 g, 0.001 mol) to obtain the title compound (0.11 g, yield: 23%).

¹H-NMR (300 MHz, DMSO-d₆) δ 2.10 (s, 6H), 3.28 (s, 2H), 5.73 (dd, 1H), 6.24 (dd, 1H), 6.33 (dd, 1H), 6.80 (dd, 1H), 7.00 (t, 1H), 7.15 (m, 2H), 7.32 (m, 4H), 7.56 (d, 2H), 8.69 (d, 1H), 9.70 (s, 1H), 10.22 (s, 1H).

### Example 4

### Preparation of N-(3-(2-(4-(4-methylpiperazin-1-carbonyl)phenylamino)-[1,2,4]triazolo[1,5-a]pyridin-8-yloxy)phenyl)acrylamide

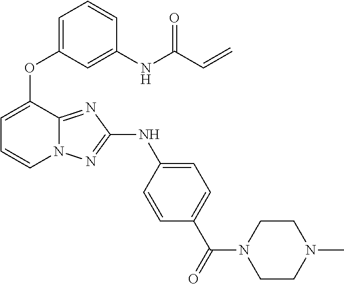 [SMILES: C=CC(=O)Nc1cccc(Oc2cccn3nc(Nc4ccc(C(=O)N5CCN(C)CC5)cc4)nc23)c1]

The procedures of Steps 5, 6 and 7 of Example 1 were repeated in sequence, except for using the compound obtained in Step 4 (0.35 g, 0.001 mol) and (4-aminophenyl)(4-methylpiperazin-1-yl)methanone (0.23 g, 0.001 mol) to obtain the title compound (0.13 g, yield: 25%).

¹H-NMR (300 MHz, DMSO-d₆) δ 2.19 (s, 3H), 2.30 (m, 4H), 3.48 (m, 4H), 5.74 (dd, 1H), 6.22 (dd, 1H), 6.37 (dd, 1H), 6.80 (d, 1H), 7.04 (t, 1H), 7.32 (m, 4H), 7.41 (m, 2H), 7.66 (d, 2H), 8.71 (d, 1H), 10.21 (s, 1H), 10.29 (s, 1H).

### Example 5

### Preparation of N-(3-(2-(4-(4-isopropylpiperazin-1-yl)phenylamino)-[1,2,4]triazolo[1,5-a]pyridin-8-yloxy)phenyl)acrylamide

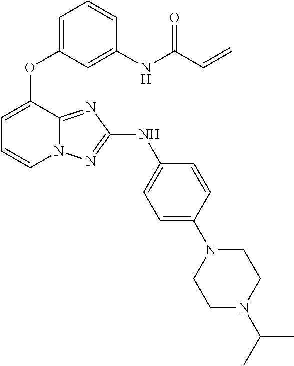 [SMILES: C=CC(=O)Nc1cccc(Oc2cccn3nc(Nc4ccc(N5CCN(C(C)C)CC5)cc4)nc23)c1]

The procedures of Steps 5, 6 and 7 of Example 1 were repeated in sequence, except for using the compound obtained in Step 4 (0.37 g, 0.001 mol) and 4-(4-isopropylpiperazin) (0.24 g, 0.001 mol) to obtain the title compound (0.15 g, yield: 27%).

¹H-NMR (300 MHz, DMSO-d₆) δ 1.02 (d, 6H), 2.50 (s, 3H), 2.60 (m, 4H), 3.02 (m, 4H), 3.40 (m, 1H), 5.73 (dd, 1H), 6.23 (dd, 1H), 6.33 (m, 1H), 6.89 (m, 4H), 7.38 (m, 5H), 8.64 (d, 1H), 9.41 (s, 1H), 10.19 (s, 1H).

### Preparation Example 1

### Preparation of Tablet

According to a conventional method, single tablets for oral administration comprising each of the compounds of formula (I) obtained in Examples 1 to 5 as an active ingredient were prepared based on the composition and amount shown in Table 1.

TABLE 1

| Composition | Amount/tablet |
|---|---|
| Active ingredient | 100 mg |
| Corn starch | 80 mg |
| Lactose | 80 mg |
| Magnesium stearate | 5 mg |

### Preparation Example 2

### Preparation of Capsule

According to a conventional method, hard gelatin capsules for oral administration comprising each of the compounds of formula (I) obtained in Examples 1 to 5 as an active ingredient were prepared based on the composition and amount shown in Table 2.

TABLE 2

| Composition | Amount/capsule |
|---|---|
| Active ingredient | 100 mg |
| Corn starch | 40 mg |
| Lactose | 80 mg |
| Crystalline cellulose | 80 mg |
| Magnesium stearate | 5 mg |

### Preparation Example 3

### Preparation of Injectable Formulation

According to a conventional method, injectable formulations comprising each of the compounds of formula (I) obtained in Examples 1 to 5 as an active ingredient were prepared based on the composition and amount shown in Table 3, wherein when a salt of the compound of formula (I) was used, the pH value was not adjusted.

TABLE 3

| Composition | Amount/injectable formulation | |
|---|---|---|
| Active ingredient | 20 | mg |
| 5% glucose solution | 10 | mL |

| | |
|---|---|
| HCl (1N) | q.s. to pH 4 |

### Preparation Example 4

### Preparation of Injectable Formulation

According to a conventional method, injectable formulations comprising each of the compounds of formula (I) obtained in Examples 1 to 5 as an active ingredient were prepared based on the composition and amount shown in Table 4.

TABLE 4

| Composition | Amount/injectable formulation | |
|---|---|---|
| Active ingredient | 20 | mg |
| Polyethylene glycol 400 | 2 | mL |
| Sterile water | 8 | mL |

### Test Example 1

### Evaluation of JAK3 and BTK Inhibitory Activity

The compounds prepared in Examples 1 to 5 were tested for JAK3 and BTK kinase inhibitory activity. Kinase inhibitory activity was measured by using Z-Lyte Kinase Assay Kit (Invitrogen), and JAK3 and BTK enzymes were purchased from Invitrogen (PV3855, PV3190).

Specifically, the compounds of Examples 1 to 5 were diluted with an aqueous solution of 4% DMSO to obtain solutions with concentrations in the range of 1 to 0.0001 μM. Each kinase was diluted to 1 to 10 ng/assay, and ATP was diluted to form a kinase buffer (50 mM HEPES, pH 7.4; 10 mM MgCl₂; 1 mM EGTA; and 0.01% BRIJ-35) by calculating an approximate Kd value. The assays were performed in 384-well polystyrene flat-bottomed plates. Peptide substrate having a suitable concentration, 10 μL of mixed kinase solution and 5 μL of ATP solution having a concentration of 5 to 300 μM were added to 5 μL of the diluted solution of the compound, and allowed to react in a mixer for 60 minutes at room temperature. After 60 minutes, 10 μL of fluorescent labeling reagents was added to each mixture so as to allow fluorescent labeling of peptide substrates, followed by adding a finishing solution to complete the reaction. The fluorescence level was determined with a Molecular Device at 400 nm (excitation filter) and 520 nm (emission filter). The kinase inhibitory activities of the compounds were calculated in phosphorylation rates between 0˜400% against the control group (staurosporine or each of kinase inhibitor) according to the reference protocol of the kit, and percentage inhibition was determine and plotted against concentration (x-axis) to calculate 50% inhibitory concentration (IC₅₀). The calculation and analysis of IC₅₀ were carried out by using Microsoft Excel. The results are shown in Table 5.

In Table 5, A means IC₅₀≦100 nM; B means IC₅₀=100˜500 nM; C means IC₅₀=500˜1,000 nM; and D means IC₅₀≧1,000 nM.

TABLE 5
IC₅₀

| Example | JAK3 | BTK |
|---|---|---|
| 1 | A (9 nM) | A (73 nM) |
| 2 | A | B |
| 3 | A | B |
| 4 | A | B |
| 5 | A | C |

What is claimed is:

1. A compound of formula (I) or a pharmaceutically acceptable salt thereof: 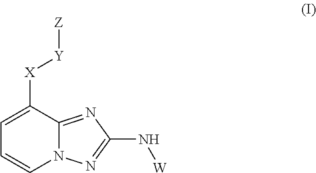
wherein,
X is O, NH, CH₂, S, SO or SO₂;
Y is phenyl or pyridyl;
Z is 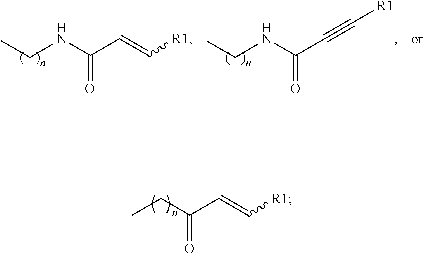
n is an integer ranging from 0 to 4;
R1 is each independently hydrogen, C₁₋₆alkoxy or di(C₁₋₆alkyl)aminomethyl; and
W is phenyl, pyridyl, or phenyl substituted with one or more substituents selected from the group consisting of hydrogen, halogen, hydroxy, amino, C₁₋₆alkylamino, C₁₋₆alkylheterocyclylamino, di(C₁₋₆alkyl)aminoC₁₋₆alkyl, heterocycle, hydroxy heterocycle, C₁₋₆alkylheterocycle, hydroxyC₁₋₆alkylheterocycle, C₁₋₆alkoxyC₁₋₆alkylheterocycle, heterocyclylcarbonyl, and heterocyclylC₁₋₆alkylcarbonyl, wherein the heterocycle is a saturated 3- to 8-membered monocyclic hetero ring independently containing one or more of heteroatoms selected from N, O and S.

2. The compound of claim 1, wherein W is selected from the group consisting of:

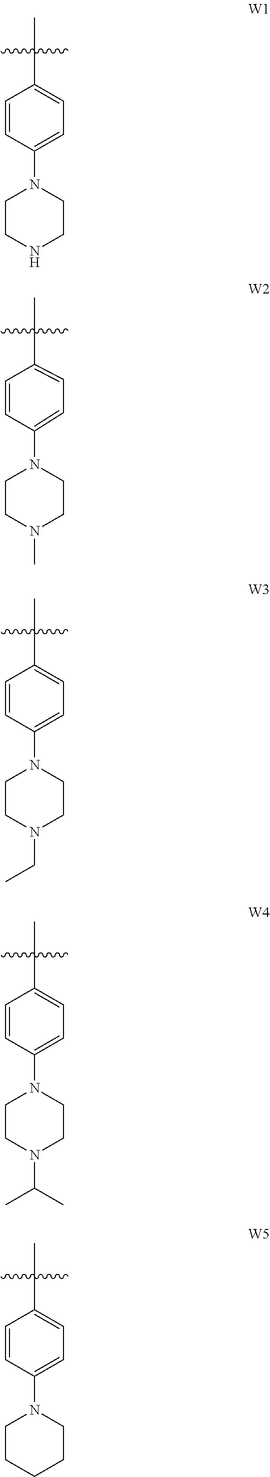 [SMILES: *c1ccc(N2CCNCC2)cc1 ; *c1ccc(N2CCN(C)CC2)cc1 ; *c1ccc(N2CCN(CC)CC2)cc1 ; *c1ccc(N2CCN(C(C)C)CC2)cc1 ; *c1ccc(N2CCCCC2)cc1]

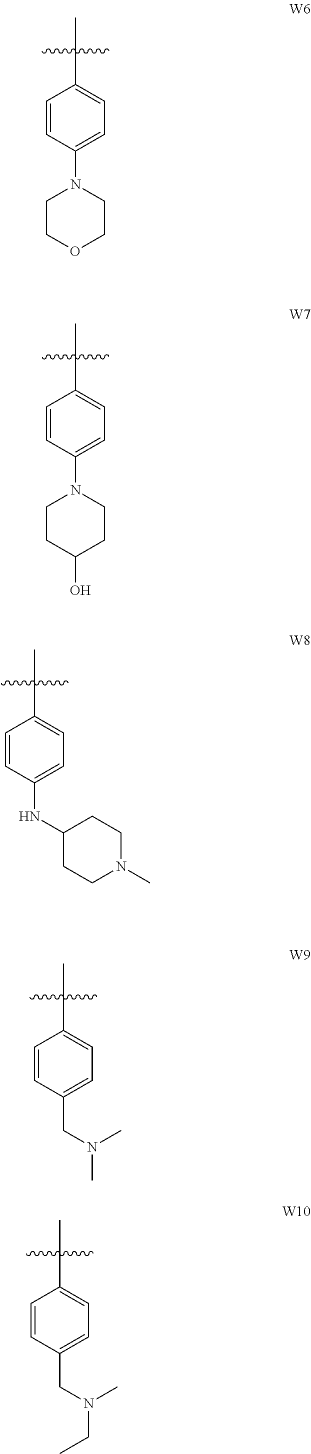 [SMILES: *c1ccc(N2CCOCC2)cc1 ; *c1ccc(N2CCC(O)CC2)cc1 ; *c1ccc(NC2CCN(C)CC2)cc1 ; *c1ccc(CN(C)C)cc1 ; *c1ccc(CN(C)CC)cc1]

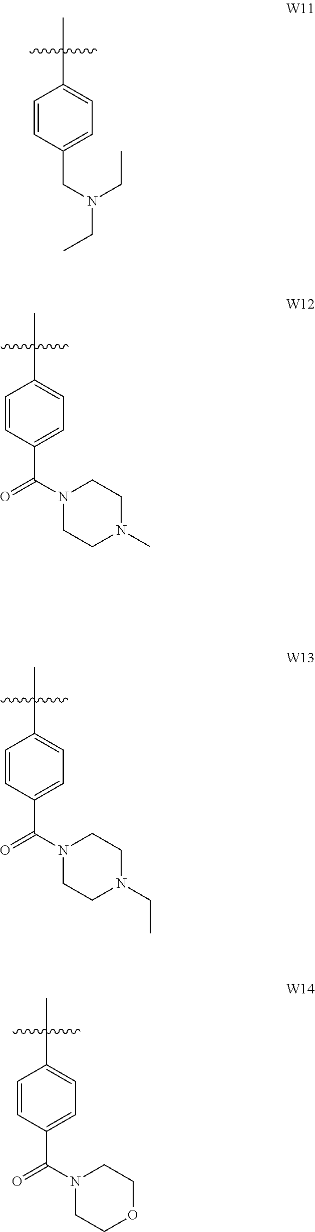 [SMILES: *c1ccc(CN(CC)CC)cc1 ; *c1ccc(C(=O)N2CCN(C)CC2)cc1 ; *c1ccc(C(=O)N2CCN(CC)CC2)cc1 ; *c1ccc(C(=O)N2CCOCC2)cc1]

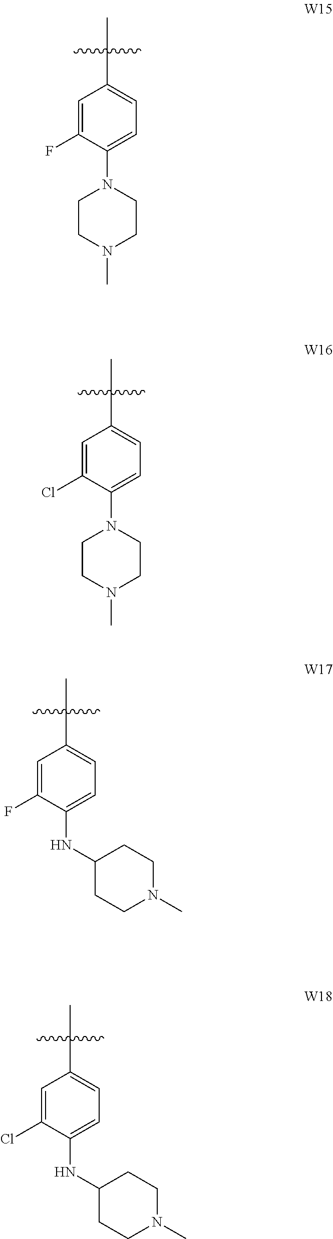 [SMILES: *c1ccc(N2CCN(C)CC2)c(F)c1 ; *c1ccc(N2CCN(C)CC2)c(Cl)c1 ; *c1ccc(NC2CCN(C)CC2)c(F)c1 ; *c1ccc(NC2CCN(C)CC2)c(Cl)c1]

3. The compound of claim 1, which is selected from the group consisting of:
N-(3-(2-(4-(4-methylpiperazin-1-yl)phenylamino)-[1,2,4]triazolo[1,5-a]pyridin-8-yloxy)phenyl)acrylamide;
N-(3-(2-(3-fluoro-4-(1-methylpiperidin-4-ylamino)phenylamino)-[1,2,4]triazolo[1,5-a]pyridin-8-yloxy)phenyl)acrylamide;
N-(3-(2-(4-((dimethylamino)methyl)phenylamino)-[1,2,4]triazolo[1,5-a]pyridin-8-yloxy)phenyl)acrylamide;
N-(3-(2-(4-(4-methylpiperazin-1-carbonyl)phenylamino)-[1,2,4]triazolo[1,5-a]pyridin-8-yloxy)phenyl)acrylamide; and
N-(3-(2-(4-(4-isopropylpiperazin-1-yl)phenylamino)-[1,2,4]triazolo[1,5-a]pyridin-8-yloxy)phenyl)acrylamide.

4. A pharmaceutical composition for preventing or treating inflammatory diseases, autoimmune diseases, proliferative diseases or hyperproliferative diseases, immunologically mediated diseases, cancers, or tumors, which comprises the compound of formula (I) or its pharmaceutically acceptable salt of claim 1 as an active ingredient.

5. The pharmaceutical composition of claim 4, wherein the inflammatory diseases, autoimmune diseases, proliferative diseases or hyperproliferative diseases, immunologically mediated diseases, cancers, or tumors are mediated by one or more of kinases selected from the group consisting of: Janus kinase 3 (JAK3), Bruton's tyrosine kinase (BTK), IL-2 inducing T-cell kinase (ITK), resting lymphocyte kinase (RLK) and bone marrow tyrosine kinase (BMX).

6. The pharmaceutical composition of claim 4, wherein the inflammatory disease, autoimmune disease, proliferative diseases or hyperproliferative diseases, immunologically mediated diseases, cancers, or tumors are mediated by abnormally activated T-lymphocytes, B-lymphocytes or both.

7. The pharmaceutical composition of claim 4, wherein the inflammatory diseases, autoimmune diseases, proliferative diseases or hyperproliferative diseases, or immunologically mediated diseases are selected from the group consisting of: arthritis, rheumatoid arthritis, spondyloarthropathy, gouty arthritis, osteoarthritis, juvenile arthritis, other arthritic conditions, lupus, systemic lupus erythematosus (SLE), skin-related diseases, psoriasis, eczema, dermatitis, atopic dermatitis, pain, pulmonary disorder, lung inflammation, adult respiratory distress syndrome (ARDS), pulmonary sarcoidosis, chronic pulmonary inflammatory disease, chronic obstructive pulmonary disease (COPD), cardiovascular disease, artherosclerosis, myocardial infarction, congestive heart failure, cardiac reperfusion injury, inflammatory bowel disease, Crohn's disease, ulcerative colitis, irritable bowel syndrome, asthma, Sjogren's syndrome, autoimmune thyroid disease, urticaria, multiple sclerosis, scleroderma, allograft rejection, xenotransplantation, idiopathic thrombocytopenic purpura (ITP), Parkinson's disease, Alzheimer's disease, diabetic associated disease, inflammation, pelvic inflammatory disease, allergic rhinitis, allergic bronchitis, allergic sinusitis, leukemia, lymphoma, B-cell lymphoma, T-cell lymphoma, myeloma, acute lymphoid leukemia (ALL), chronic lymphoid leukemia (CLL), acute myeloid leukemia (AML), chronic myeloid leukemia (CML), hairy cell leukemia, Hodgkin's disease, non-Hodgkin's lymphoma, multiple myeloma, myelodysplastic syndrome (MDS), myeloproliferative neoplasms (MPN), diffuse large B-cell lymphoma and follicular lymphoma.

8. The pharmaceutical composition of claim 4, which further comprises another anticancer agent selected from the group consisting of cell signal transduction inhibitors, mitosis inhibitors, alkylating agents, anti-metabolites, intercalating agents, topoisomerase inhibitors, immunotherapeutic agents, anti-hormonal agents and a mixture thereof as an active ingredient.

9. The pharmaceutical composition of claim 4, which further comprises an additional drug selected from the group consisting of steroids, methotrexate, lefluonomide, anti-TNF α agents, calcineurin inhibitors, anti-histamines and a mixture thereof as an active ingredient.

10. A method for preventing or treating inflammatory diseases, autoimmune diseases, proliferative diseases or hyperproliferative diseases, immunologically mediated diseases, cancers, or tumors in an animal, comprising the step of administering to the animal an effective amount of the compound of formula (I) or its pharmaceutically acceptable salt of claim 1.

11. A use of the compound of formula (I) or its pharmaceutically acceptable salt of claim 1 for the manufacture of a medicament for preventing or treating inflammatory diseases, autoimmune diseases, proliferative diseases or hyperproliferative diseases, immunologically mediated diseases, cancers, or tumors.
